(19) United States
(12) Patent Application Publication
(10) Pub. No.: US 2011/0028921 A1
(43) Pub. Date: Feb. 3, 2011

(54) PORTABLE WOUND THERAPY APPARATUS AND METHOD

(75) Inventors: Edward Yerbury Hartwell, York (GB); Jonathan Chappel, York (GB)

Correspondence Address: KNOBBE MARTENS OLSON & BEAR LLP, 2040 MAIN STREET, FOURTEENTH FLOOR, IRVINE, CA, 92614, US

(73) Assignee: SMITH & NEPHEW PLC, LONDON (UK)

(21) Appl. No.: 12/808,547

(22) PCT Filed: Dec. 11, 2008
(86) PCT No.: PCT/GB2008/004094

§ 371 Date: Oct. 13, 2010

(30) Foreign Application Priority Data
Dec. 18, 2007 (GB) 0724564.0

(51) Int. Cl. A61M 1/00

(52) U.S. Cl. 604/320; 604/319

(57) ABSTRACT

Methods and apparatuses are disclosed for providing topical negative pressure to a wound. One such apparatus comprises a waste canister for collecting and holding wound exudate and at least one pump arranged to urge wound exudate along a flow path from the wound to the waste canister. The pump can be further configured to deliver a single factory set pressure. Accordingly, the pressure delivered to the wound would be determined by the pressure set for the pump along with the parameters associated with the flow path, such as whether a leak or a blockage occurs.

The present invention relates to apparatus and a method for the application of topical negative pressure (TNP) therapy to wounds. In particular, but not exclusively, the present invention relates to a method and apparatus for providing topical negative pressure at a wound site using a disposable low cost device.

There is much prior art available relating to the provision of apparatus and methods of use thereof for the application of TNP therapy to wounds together with other therapeutic processes intended to enhance the effects of the TNP therapy. Examples of such prior art include those listed and briefly described below.

TNP therapy assists in the closure and healing of wounds by reducing tissue oedema; encouraging blood flow and granulation of tissue; removing excess exudates and may reduce bacterial load and thus, infection to the wound. Furthermore, TNP therapy permits less outside disturbance of the wound and promotes more rapid healing.

In International patent application, WO 2004/037334, apparatus, a wound dressing and a method for aspirating, irrigating and cleansing wounds are described. In very general terms, this invention describes the treatment of a wound by the application of topical negative pressure (TNP) therapy for aspirating the wound together with the further provision of additional fluid for irrigating and/or cleansing the wound, which fluid, comprising both wound exudates and irrigation fluid, is then drawn off by the aspiration means and circulated through means for separating the beneficial materials therein from deleterious materials. The materials which are beneficial to wound healing are recirculated through the wound dressing and those materials deleterious to wound healing are discarded to a waste collection bag or vessel.

In International patent application, WO 2005/04670, apparatus, a wound dressing and a method for cleansing a wound using aspiration, irrigation and cleansing wounds are described. Again, in very general terms, the invention described in this document utilises similar apparatus to that in WO 2004/037334 with regard to the aspiration, irrigation and cleansing of the wound, however, it further includes the important additional step of providing heating means to control the temperature of that beneficial material being returned to the wound site/dressing so that it is at an optimum temperature, for example, to have the most efficacious therapeutic effect on the wound.

In International patent application, WO 2005/105180, apparatus and a method for the aspiration, irrigation and/or cleansing of wounds are described. Again, in very general terms, this document describes similar apparatus to the two previously mentioned documents hereinabove but with the additional step of providing means for the supply and application of physiologically active agents to the wound site/dressing to promote wound healing.

The content of the above references is included herein by reference.

However, the above apparatus and methods are generally only applicable to a patient when hospitalised as the apparatus is complex, needing people having specialist knowledge in how to operate and maintain the apparatus, and also relatively heavy and bulky, not being adapted for easy mobility outside of a hospital environment by a patient, for example.

Some patients having relatively less severe wounds which do not require continuous hospitalisation, for example, but whom nevertheless would benefit from the prolonged application of TNP therapy, could be treated at home or at work subject to the availability of an easily portable and maintainable TNP therapy apparatus.

GB-A-2 307 180 describes a portable TNP therapy unit which may be carried by a patient clipped to belt or harness. However, the therapy unit includes many costly parts and involves a complex manufacturing process to produce. Also control of the pressure provided by the unit is complex and prone to user error.

It is an aim of the present invention to at least partly mitigate the above-mentioned problems.

It is an aim of embodiments of the present invention to provide a wound therapy device which can be manufactured simply and efficiently and which is simple to understand for a user and thus is not prone to user error.

It is an aim of embodiments of the present invention to provide apparatus and a method for providing topical negative pressure at a wound site in which only a single factory set pressure is provided by the apparatus. In this way a faulty user interface or faulty input on behalf of a user cannot cause problems to a patient.

It is an aim of embodiments of the present invention to provide a disposable apparatus for providing topical negative pressure using relatively cheap to produce and assemble components. This enables the apparatus to be substantially disposable after a single use by a user. As a result contamination problems can be obviated.

According to a first aspect of the present invention there is provided apparatus for providing topical negative pressure at a wound site; comprising;
a waste canister for collecting and holding wound exudate material; and
at least one pump element arranged to urge wound exudate along a flow path from a wound site to the waste canister; wherein
the pump element delivers only a single factory set pressure.

The invention is comprised in part of an overall apparatus for the provision of TNP therapy to a patient in almost any environment. The apparatus is lightweight, may be battery powered by a rechargeable battery pack contained within a device (henceforth, the term “device” is used to connote a unit which may contain all of the power supply, power supply recharging, indicator means and means for initiating and sustaining aspiration functions to a wound and any further necessary functions of a similar nature). When outside the home, for example, the apparatus may provide for an extended period of operation on battery power and in the home, for example, the device or batteries may be connected to the mains by a charger unit for recharging whilst still being used and operated by the patient.

The overall apparatus of which the present invention is a part comprises: a dressing covering the wound and sealing at least an open end of an aspiration conduit to a cavity formed over the wound by the dressing; an aspiration tube comprising at least one lumen therethrough leading from the wound dressing to a waste material canister for collecting and holding wound exudates/waste material prior to disposal; and, a power, control and aspiration initiating and sustaining device associated with the waste canister.

The dressing covering the wound may be any type of dressing normally employed with TNP therapy and, in very general terms, may comprise, for example, a semi-permeable, flexible, self-adhesive drape material, as is known in the dressings art, to cover the wound and seal with surrounding sound tissue to create a sealed cavity or void over the wound. There may aptly be a porous barrier and support member in the cavity between the wound bed and the covering material to enable an even vacuum distribution to be achieved over the area of the wound. The porous barrier and support member being, for example, a foam or known wound contact type material resistant to crushing under the levels of vacuum created and which permits transfer of wound exudates across the wound area to the aspiration conduit sealed to the flexible cover drape over the wound.

The aspiration conduit may be a plain flexible tube, for example, having a single lumen therethrough and made from a plastics material compatible with raw tissue, for example. However, the aspiration conduit may have a plurality of lumens therethrough to achieve specific objectives relating to the invention. A portion of the tube sited within the sealed cavity over the wound may have a structure to enable continued aspiration and evacuation of wound exudates without becoming constricted or blocked even at the higher levels of the negative pressure range envisaged.

It is envisaged that the negative pressure provided by the apparatus embodying the present invention may be between about −50 mmHg and −200 mmHg (note that these pressures are relative to normal ambient atmospheric pressure thus, −200 mmHg would be about 560 mmHg in practical terms). Aptly, the pressure may be between about −75 mmHg and −150 mmHg. Alternatively a pressure of upto −75 mmHg, upto —80 mmHg or over −80 mmHg can be used. Also aptly a pressure of below −75 mmHg could be used. Alternatively a pressure of over −100 mmHg could be used or over −150 mmHg.

The aspiration conduit at its distal end remote from the dressing may be attached to the waste canister at an inlet port or connector. The device containing the means for initiating and sustaining aspiration of the wound/dressing may be situated between the dressing and waste canister, however, in a preferred embodiment of the apparatus embodying the present invention, the device may aspirate the wound/dressing via the canister thus, the waste canister may preferably be sited between the wound/dressing and device.

The aspiration conduit at the waste material canister end may preferably be bonded to the waste canister to prevent inadvertent detachment when being caught on an obstruction, for example.

The canister may be a plastics material moulding or a composite unit comprising a plurality of separate mouldings. The canister may aptly be translucent or transparent in order to visually determine the extent of filling with exudates. However, the canister and device may in some embodiments provide automatic warning of imminent canister full condition and may also provide means for cessation of aspiration when the canister reaches the full condition.

The canister may be provided with filters to prevent the exhaust of liquids and odours therefrom and also to prevent the expulsion of bacteria into the atmosphere. Such filters may comprise a plurality of filters in series. Examples of suitable filters may comprise hydrophobic filters of 0.2 μm pore size, for example, in respect of sealing the canister against bacteria expulsion and 1 μm against liquid expulsion.

Aptly, the filters may be sited at an upper portion of the waste canister in normal use, that is when the apparatus is being used or carried by a patient the filters are in an upper position and separated from the exudate liquid in the waste canister by gravity. Furthermore, such an orientation keeps the waste canister outlet or exhaust exit port remote from the exudate surface.

Aptly the waste canister may be filled with an absorbent gel such as ISOLYSEL (trade mark), for example, as an added safeguard against leakage of the canister when full and being changed and disposed of. Added advantages of a gel matrix within the exudate storing volume of the waste canister are that it prevents excessive movement, such as slopping, of the liquid, minimises bacterial growth and minimises odours.

The waste canister may also be provided with suitable means to prevent leakage thereof both when detached from the device unit and also when the aspiration conduit is detached from the wound site/dressing.

The canister may have suitable means to prevent emptying by a user (without tools or damage to the canister) such that a full or otherwise end-of-life canister may only be disposed of with waste fluid still contained.

The device and waste canister may have mutually complementary means for connecting a device unit to a waste canister whereby the aspiration means in the device unit automatically connects to an evacuation port on the waste canister such that there is a continuous aspiration path from the wound site/dressing to an exhaust port on the device.

Aptly, the exhaust port from the fluid path through the apparatus is provided with filter means to prevent offensive odours from being ejected into the atmosphere.

In general terms the device unit comprises an aspirant pump, a silencer, a canister for collecting and holding waste exudate, one or more filters and a user interface for stopping and starting of the apparatus by a user. The device unit may contain all of the above features within a single unified casing or within separate units which are connected together to form the device.

In view of the fact that the device unit contains the majority of the intrinsic equipment cost therein ideally it will also be able to survive impact.

In terms of pressure capability the aspiration means may be able to apply a maximum pressure drop of at least −200 mmHg to a wound site/dressing.

Parameters of the pump used are factory set to prevent the minimum pressure achieved from exceeding for example −200 mmHg so as not to cause undue patient discomfort. The pressure provided by the unit is determined at the factory at a discreet level such as −50, −75, −100, −125, −150, −175 mmHg, for example, depending upon the needs of the wound in question and the advice of a clinician. Thus a deliverable pressure in use may be from −25 to −80 mmHg, or −50 to −76 mmHg, or −50 to −75 mmHg as examples.

The apparatus of the present invention may be provided with a carry case and suitable support means such as a shoulder strap or harness, for example. The carry case may be adapted to conform to the shape of the apparatus comprised in the joined together device and waste canister. In particular, the carry case may be provided with a bottom opening flap to permit the waste canister to be changed without complete removal of the apparatus form the carry case.

The carry case may be provided with an aperture covered by a displaceable flap to enable user access to one or more buttons or displays of a user interface to select the therapy applied by the apparatus.

According to a second aspect of the present invention, there is provided a method for the topical negative pressure treatment of a wound using the apparatus of the first aspect.

According to a third aspect of the present invention there is provided a method for providing topical negative pressure at a wound site, comprising the steps of:
disposing at least one pump element in a flow path for fluid communication with a wound site;
delivering only a single factory set pressure from the pump element thereby urging wound exudate along the flow path from the wound site to a waste canister; and
collecting wound exudate in the waste canister.

In order that the present invention may be more fully understood, examples will now be described by way of illustration only with reference to the accompanying drawings, of which:

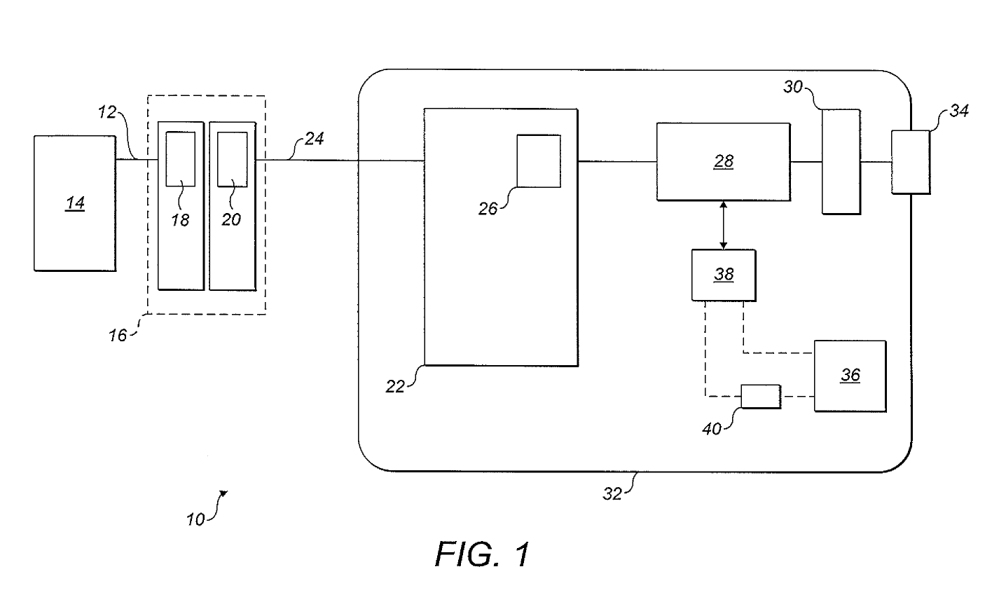

FIG. 1 shows a generalised schematic block diagram showing a general view of an apparatus and the constituent apparatus features thereof;

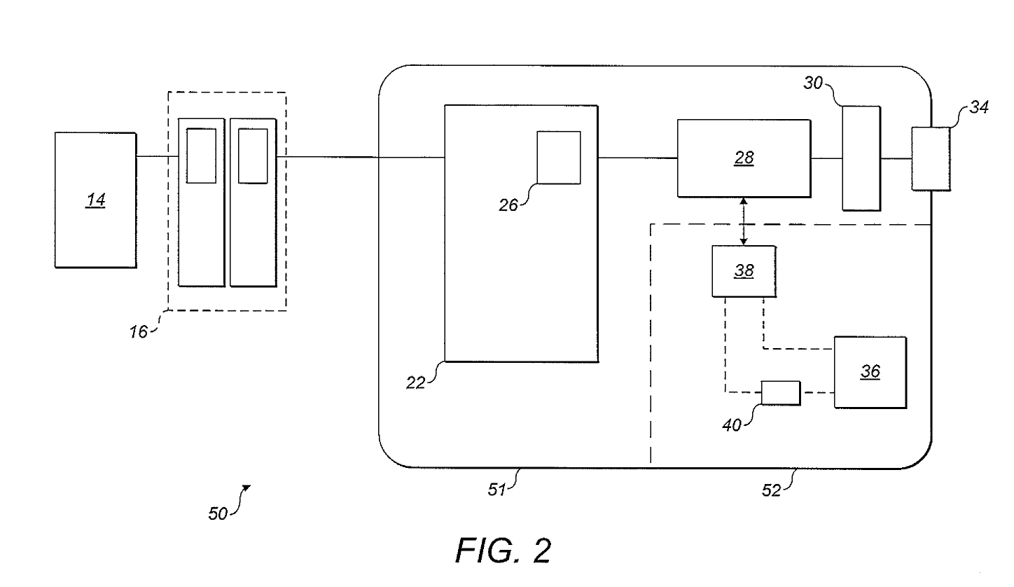

FIG. 2 illustrates a device in which a battery and pump drive unit are detachable;

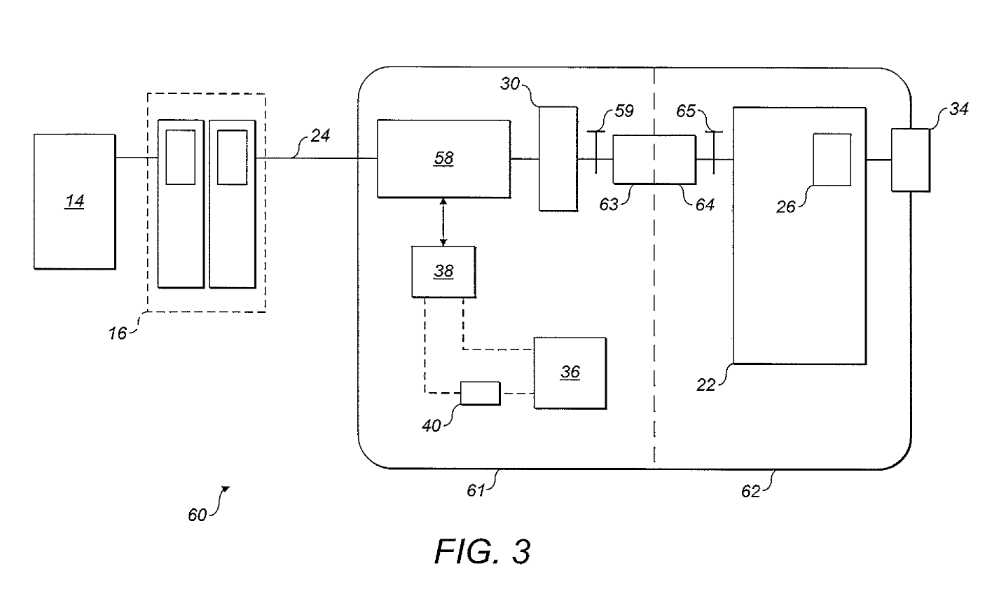

FIG. 3 illustrates a device including two integral units; and

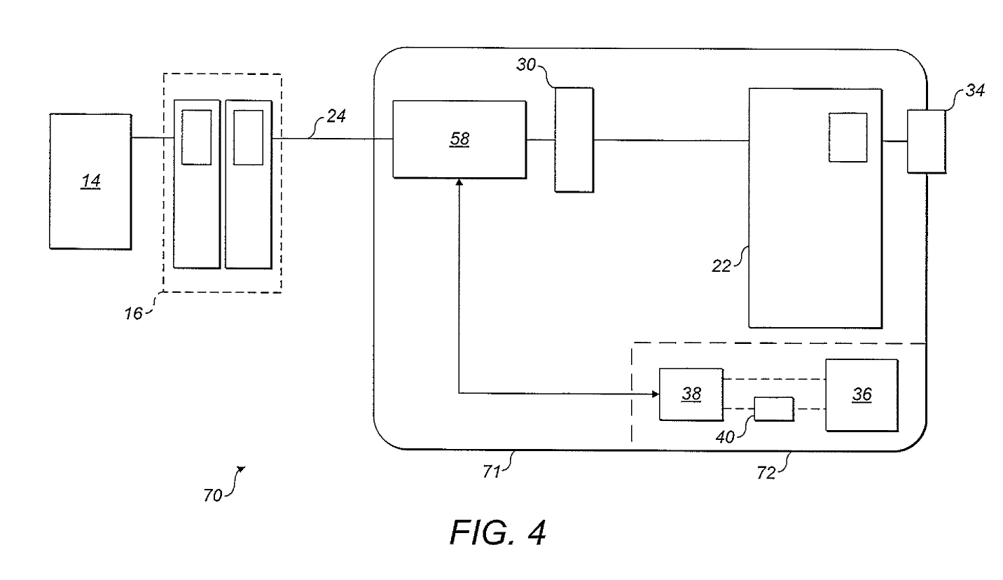

FIG. 4 illustrates a device including two integral units.

Referring now to FIGS. 1 to 4 of the drawings and where the same or similar features are denoted by common reference numerals.

FIG. 1 shows a generalised schematic view of an apparatus 10 of a portable topical negative pressure (TNP) system. It will be understood that embodiments of the present invention are generally applicable to use in such a TNP system. Briefly, negative pressure wound therapy assists in the closure and healing of many forms of “hard to heal” wounds by reducing tissue oedema; encouraging blood flow and granular tissue formation; removing excess exudate and may reduce bacterial load (and, therefore, infection). In addition the therapy allows for less disturbance of a wound leading to more rapid healing. The TNP system is detailed further hereinafter but in summary includes a portable body including a canister and a pump capable of providing an extended period of continuous therapy. The system is connected to a patient via a length of tubing with an end of the tubing operably secured to a wound dressing on the patient.

More particularly, as shown in FIG. 1, the apparatus comprises an aspiration conduit 12 operably and an outer surface thereof at one end sealingly attached to a dressing 14. The dressing 14 will not be further described here other than to say that it is formed in a known manner from well know materials to those skilled in the dressings art to create a sealed cavity over and around a wound to be treated by TNP therapy with the apparatus of the present invention. The aspiration conduit has an in-line connector 16 comprising connector portions 18, 20 intermediate its length between the dressing 14 and a waste canister 22. The aspiration conduit between the connector portion 20 and the canister 22 is denoted by a different reference numeral 24 although the fluid path through conduit portions 12 and 24 to the waste canister is continuous. The connector portions 18, 20 join conduit portions 12, 24 in a leak-free but disconnectable manner. Alternatively a single conduit between the dressing and canister may be used without connectors. The waste canister 22 is provided with one or more filters 26 which prevent the escape via an exit port of liquid and bacteria from the waste canister. The filters may comprise a 1 μm hydrophobic liquid filter and a 0.2 μm bacteria filter such that all liquid and bacteria is confined to an interior waste collecting volume of the waste canister 22. The exit port of the waste canister is connected to an aspirant pump 28. The aspiration path takes the aspirated fluid which in the case of fluid on the exit side of exit port from the filter 28 is gaseous through a silencer system 30 and an optional final filter having an activated charcoal matrix which ensures that no odours escape with the gas exhausted from the device 32 via an exhaust port 34. The filter material also serves as noise reducing material to enhance the effect of the silencer system 30. The device 32 also contains a battery pack 36 to power the apparatus which battery pack powers a pump drive unit 38 for the pump 28. Aptly the one or more battery is rechargeable. A switch 40 forms part of a user interface to enable a user to switch the pump on and/or off.

The wound exudate with air is aspirated from the wound site/dressing 14 via the conduit 12, the two connector portions 18, 20 and the conduit 24 into the waste canister 22. The waste canister 22 comprises a relatively large volume in the region of 500 ml into which exudate from the wound is drawn by the aspiration system. The fluid drawn into the canister volume is a mixture of both air drawn into the dressing 14 via the semi-permeable adhesive sealing drape (not shown) and liquid in the form of wound exudates. The volume within the canister is also at a lowered pressure and the gaseous element of the aspirated fluids is exhausted from the canister volume via the filters 26 and the waste canister exhaust exit port as bacteria-free gas. From the exit port of the waste canister to the final exhaust port 54 the fluid is gaseous only.

A problem with known vacuum devices for negative pressure wound therapy (NPWT) is that they are expensive to purchase and are complex electronic devices capable of running both on mains and battery power. The cost is prohibitive to many users resulting in many patients receiving sub optimum care. Embodiments of the present invention overcome such problems by doing away with much of the complexity of prior art devices to provide a safe, cheap suction device. Complex electronics and software control are obviated and a mechanical pump system is provided that delivers a single factory set pressure. The pressure provided by the device is set at the factory by determining one or more parameters associated with the pump 28 which is utilised. For example, as shown in FIG. 1, the pump used can be a diaphragm pump. It is well known that such pumps have associated with them a valve leak back pressure, swept volume to dead volume ratio of a pumping chamber, a rate of reciprocation, strength of a diaphragm membrane and/or leak through pressure of valves. Other parameters of the pump are also known. When the diaphragm pump is designed and manufactured the parameters of the design are determined so that the diaphragm pump will provide a predetermined factory set pressure. Subsequent to manufacture there is no control provided at the apparatus 32 to vary the provided pressure. Rather the pressure delivered at the dressing 14 will be determined by the pressure set for the apparatus together with parameters associated with the flow path such as whether a leak or blockage occurs. In this way a user cannot accidentally and erroneously set an undesired pressure. Also electronics and/or software which might otherwise be needed together with feed back to control pump pressure are unnecessary, thus reducing costs and decreasing the likelihood of device failure.

By varying the parameters of the pump during design, it is possible to produce a pump that delivers a predefined maximum negative pressure. The device thus delivers a factory set pressure and can be made cheaply, simply and robustly for a single patient use.

FIG. 2 illustrates an alternative embodiment of the present invention in which, rather than the single integral unit of the device 32 shown in FIG. 1, the apparatus 50 is formed as a first integral unit 51 and a further integral unit 52. In this embodiment the canister 22, pump 28 and silencer 30 are integrally formed in a single unit. A motor drive unit 38 and battery 36 are likewise formed in a separate unit 52 which may be releasably connected to the first unit. In this way disposable sterile canisters/pumps may be made to deliver a preset maximum negative pressure that attach to a standard drive/battery pack. The connection of the drive and power source to the pump and canister may be by means of any known method, for example mechanical/magnetic etc.

FIG. 3 illustrates an alternative embodiment of the present invention in which a vane pump 58 is utilised. These types of pump are able to handle mixtures of liquids and gases and solids so are suitable for directly pumping wound fluid. The pump 58 is thus placed in the fluid path such that an inlet connects fluidically to a wound via the conduit 24. It is to be noted by contrast that the embodiments illustrated in FIGS. 1 and 2 which use a diaphragm pump utilise the pump downstream of the canister and filter and thus are required only to handle gaseous fluids. The outlet of the pump 58 is connected to a silencer and via a valve 59 to a connector. The apparatus 60 includes a first unit 61 and further unit 62 which are connected together. Connection may be via known techniques such as mechanical/magnetic etc techniques and may include a flow line connector 63 downstream of the valve 59. A mating connector 64 is connectable in a flow path with a further valve 65 disposed between the connector 64 and a canister 22. The canister 22 acts as a liquid/solid trap such that gas may be expelled via a hydrophobic filter 26 on the outlet of the container. Such filters will be in the range 2.0 to 0.02 microns. It will be understood that whilst reference is made throughout this description to a canister 22 it will be understood that the canister acts as a waste receptacle and thus may be either a flexible or rigid container for collecting and holding wound exudate material.

The optional odour filter (e.g. activated carbon, zeolite or the like) is illustrated in FIG. 3. Such filters may be integral in any of the embodiments of the present invention or separate or not included.

By varying parameters of the vane pump during design and manufacture, the pressure ultimately provided by the apparatus can be factory set. For example, by varying the speed of rotation, flexibility and/or geometry of the vanes and/or the eccentricity of the pump it is possible to set a maximum attainable negative pressure. These parameters are set during design and manufacture at a factory so that once built the pump offers only a single factory set pressure. The valves 59, 65 are provided to isolate the waste canister and pump to facilitate safe removal of the waste canister for disposal. Such valves may be interlocked with the latching of the canister such that the canister cannot be removed prior to closure of the valves, thus minimising risk of contamination. Optionally, only a single upstream valve 59 may be included.

Subsequent to use by a user the further integral unit 62 may be disposed of with replacement units being available for a subsequent use or user.

In an alternative embodiment of the present invention the pump and canister may be integral. As illustrated in FIG. 4 apparatus 70 is formed of a first integral unit 71 including the pump 58, silencer 30 and canister 22. A further integral unit 72 which is releasably securable to the first integral unit 71 includes the battery 36 and drive unit 38 for the pump 58. In this way a standard battery pack 72 can be used with a disposable canister and pump unit. The two parts of the apparatus 70 may be releasably secured together via known connecting methods such as magnetic or mechanical connectors.

Optionally, according to further embodiments of the present invention, a flow meter indicator may be provided to indicate presence of a leak in the dressing system. The flow meter may be positioned anywhere in the fluidic path of the system shown in FIGS. 1 to 4 although aptly the flow meter is positioned on the outlet of the pump. The flow meter may be a simple mechanical device such as a hinged flap with indicator flag/window or may be a more sophisticated electrical device such that an alert cue may be made either visibly and/or audibly to a user when an excess leak occurs.

Prior art devices tend to be very expensive due to the number of components utilised and are complex to use requiring training and large user manuals. These devices also bring with them a risk of cross contamination between patients and users as they are intended for multi patient use and are often serviced by technicians. Embodiments of the present invention overcome these problems by reducing the need for expensive component parts and complex training. This is in part due to a limited user interface including only an on/off button and possibly one or more simple visible cues to illustrate proper functioning. Also no pressure monitoring or setting is provided with the device providing only a single factory set pressure which is determined by design parameters associated with the pump utilised. Because the apparatus is disposable or at least part of the device is disposable subsequent to a single user use, further costs are reduced because the overall parts are not required to last for extended periods of use. For example, use will typically be limited to the order of days not years. Clinicians seeing a patient may thus select a desired negative pressure which is to be applied to a wound and select a device manufactured to provide that pressure. Subsequently the clinicians have peace of mind that the therapy has not been tampered with or altered.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

1. Apparatus for providing topical negative pressure at a wound site, comprising:
a waste canister for collecting and holding wound exudate material; and
at least one pump element arranged to urge wound exudate along a flow path from a wound site to the waste canister, wherein the pump element delivers only a single factory set pressure.

2. The apparatus as claimed in claim 1, wherein one or more characteristics of the pump element determine a single predefined negative pressure delivered from the pump element.

3. The apparatus as claimed in claim 1, further comprising:
a filter member downstream in the flow path from the canister; and
a silencer member downstream in the flow path from the pump element.

4. The apparatus as claimed in claim 3, further comprising at least one battery that provides power to a motor drive for the pump element.

5. The apparatus as claimed in claim 4, wherein the battery is rechargeable.

6. The apparatus as claimed in claim 4, wherein the waste canister, the pump element, the filter member and the silencer member are arranged into an integral unit with the motor drive for the pump element and the battery.

7. The apparatus as claimed in claim 4, wherein the waste canister, the pump element, the filter member and the silencer member are arranged into a first integral unit, the first integral unit being separate from but connectable to a second integral unit, the second integral unit comprising the motor drive for the pump element and the battery.

8. The apparatus as claimed in claim 6, further comprising:
a flow meter in the flow path that indicates a negative pressure in the flow path, the flow meter being in the integral unit with the pump element.

9. The apparatus as claimed in claim 2, wherein:
the pump element comprises a diaphragm pump; and
the one or more characteristics is selected from the group consisting of: valve leak back pressure, swept volume to dead volume ratio of a pumping chamber, rate of reciprocation, strength of a diaphragm membrane and value of leak through pressure.

10. The apparatus as claimed in claim 2, wherein:
the pump element comprises a vane pump; and
the one or more characteristics is selected from the group consisting of: speed of rotation, flexibility of vanes, geometry of vanes and eccentricity of the pump.

11. The apparatus as claimed in claim 10, further comprising:
a silencer member downstream in the flow path from the pump element; and
at least one on/off valve in the flow path between the silencer member and the waste canister.

12. The apparatus as claimed in claim 11 , further comprising:
a motor drive for the pump element;
a filter member downstream in the flow path from the canister; and
at least one battery, configured to provide power to the motor drive, wherein the pump element, the silencer member, the motor drive and the at least one battery are arranged into an integral unit with the valve, the canister and the filter member.

13. The apparatus as claimed in claim 11 , further comprising:
a motor drive for the pump element;
a filter member downstream in the flow path from the canister; and
at least one battery, configured to provide power to the motor drive, wherein the pump element, the silencer member, the valve, the motor drive and the at least one battery are arranged into a first integral unit, the first integral unit being separate from but connectable to a second integral unit, the second integral unit comprising the waste canister and the filter member.

14. The apparatus as claimed in claim 12, further comprising:
a flow meter in the flow path that indicates a negative pressure in the flow path, the flow meter being in the integral unit with the pump element.

15. The apparatus as claimed in claim 1, wherein the apparatus is disposable subsequent to a single user use.

16. A method for providing topical negative pressure at a wound site, comprising:
disposing at least one pump element in a flow path for fluid communication with a wound site;
delivering only a single factory set pressure from the pump element, thereby urging wound exudate along the flow path from the wound site to a waste canister; and
collecting wound exudate in the waste canister.

17. The method as claimed in claim 16, further comprising:
determining a single predefined negative pressure delivered from the pump element responsive to one or more factory set characteristics of the pump element.

18. The method as claimed in claim 16, further comprising:
arranging the waste canister, the pump element, a filter member and a silencer unit into an integral unit with a motor drive for the pump element and a battery.

19. The method as claimed in claim 16, further comprising:
arranging the waste canister, the pump element, a filter member and a silencer member into a first integral unit, the first integral unit being separate from but connectable to a second integral unit, the second integral unit comprising a motor drive for the pump element and a battery.

20. The method as claimed in claim 18, further comprising:
indicating a negative pressure in the flow path via a flow meter in the integral unit with the pump.

21. (canceled)

22. (canceled)
